(19) United States
(12) Patent Application Publication
(10) Pub. No.: US 2003/0153010 A1
(43) Pub. Date: Aug. 14, 2003

(54) Patterned biological molecules on inner surface of enclosed tubes

(76) Inventors: Reid N. Orth, Ithaca, NY (US); Harold G. Craighead, Ithaca, NY (US); Stephen W. Turner, Ithaca, NY (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/340,190

(22) Filed: Jan. 9, 2003

Related U.S. Application Data
(60) Provisional application No. 60/347,622, filed on Jan. 10, 2002.

(51) Int. Cl. G01N 33/53; C12M 1/34; B05D 3/00

(52) U.S. Cl. 435/7.5; 427/2.11; 435/287.2

(57) ABSTRACT

Biomolecular photo-based patterning methods utilize avidin-biotin technology to immobilize functional proteins on the inner surface of silica glass tubes in desired patterns. The methods are useful for nanofluidic affinity biosensor/chromatography systems and on silicon dioxide substrates for biosensor applications. The resulting patterns are optimized based on the application. A zebra shaped pattern is utilized for an affinity chromatography system.

This application claims the benefit of priority under 35 U.S.C. 119(e) to U.S. Provisional Patent Application Serial No. 60/347,622, filed Jan. 10, 2002, which is incorporated herein by reference in its entirety.

### GOVERNMENT FUNDING

The invention described herein was made with U.S. Government support under Grant Number NSF ECS-9876771 and ARPA Number MDA972-00-1-0021. The government has certain rights in the invention.

### BACKGROUND OF THE INVENTION

Many micro and nanotechnology bioassay applications such as biosensor/chromatography systems require protein patterning to operate effectively. Biological samples must be fixed in place on a desired surface. Several methods have been developed to fix such samples on glass surfaces. However, some such techniques require large quantities of the biosample. Attempts have been made to apply the samples, and then enclose them with a glass plate. Unfortunately, the adhering process used to achieve adequate sealing also produced high heat, that adversely affected such samples.

### SUMMARY

Biomolecular photo-based patterning methods utilize avidin-biotin technology to immobilize functional proteins on the inner surface of silica glass tubes in desired patterns. The methods are useful for nanofluidic affinity biosensor/chromatography systems and on silicon dioxide substrates for biosensor applications. The resulting patterns are optimized based on the application. In one embodiment, a zebra shaped pattern is utilized for an affinity chromatography system.

In one embodiment, layering above the substrates comprises the following molecules: 3-aminopropyltriethoxysilane (3-APTS), the N-hydroxysuccinimide (NHS) ester of photoactivatable biotin, NeutrAvidin, biotinylated antibody, and target antigen (bacteria, sphere, bacteria supernatant). The photoactivatable biotin covalently bound to the 3-aminopropyltriethoxysilane (3-APTS) self assembled monolayer after irradiation by 350 nm light through a chrome plated photomask. Neutr Avidin is used in part because it has four binding sites, and only one is used to anchor it in place, leaving three open to bind with molecules in solution, such as biotinylated molecules.

In further embodiments, any other light activatable molecules that can be bound to photoactivatable biotin are utilized. The advantages of these bimolecular derivitization methods are their versatility of binding any biotinylated protein and safety from exposure to denaturing UV light, pH, chemicals, or salinity. The biotinylated proteins may be immunologically specific to a desired sample. Additionally, the inner surface of enclosed vessels may be patterned without the requirement of a high temperature anodically bonded glass cover.

Fluorescently labeled primary antibodies and protein-A coated spheres and E. coli cells serve as model target antigens for the biosensor and affinity chromatography micro- and nanofluidic systems in silicon, glass, and plastic in one embodiment.

In one embodiment, by patterning biotin and avidin layers to the inner surface of a glass capillary tube, biotinylated protein patterns are subsequently adhered to the capillary tube. The binding of porous beads or antibodies offers an affinity chromatography system to take place with nanoliters of solution, over 10-250× less solution than conventional chromatography systems.

### BRIEF DESCRIPTION OF THE DRAWINGS

FIGS. 1A, 1B, 1C, 1D, 1E, 1F and 1G illustrate an example process for forming patterned biological molecules.

FIG. 2 is a block diagram example of patterned layers of a biosensor chip with antigen.

FIG. 3 is a block diagram example of patterned layers of a biosensor chip showing E.coli antibodies being tested with anti-goat antibody.

FIG. 4 illustrates an example of patterned layers in a glass tube.

FIGS. 5A, 5B and 5C illustrate the use of the glass tube in FIG. 4 in affinity chromatography.

FIG. 6 is a block diagram of a fluidic system combined with a patterned tube.

### DETAILED DESCRIPTION OF THE INVENTION

In the following description, reference is made to the accompanying drawings that form a part hereof, and in which is shown by way of illustration specific embodiments in which the invention may be practiced. These embodiments are described in sufficient detail to enable those skilled in the art to practice the invention, and it is to be understood that other embodiments may be utilized and that structural, logical and electrical changes may be made without departing from the scope of the present invention. The following description is, therefore, not to be taken in a limited sense, and the scope of the present invention is defined by the appended claims.

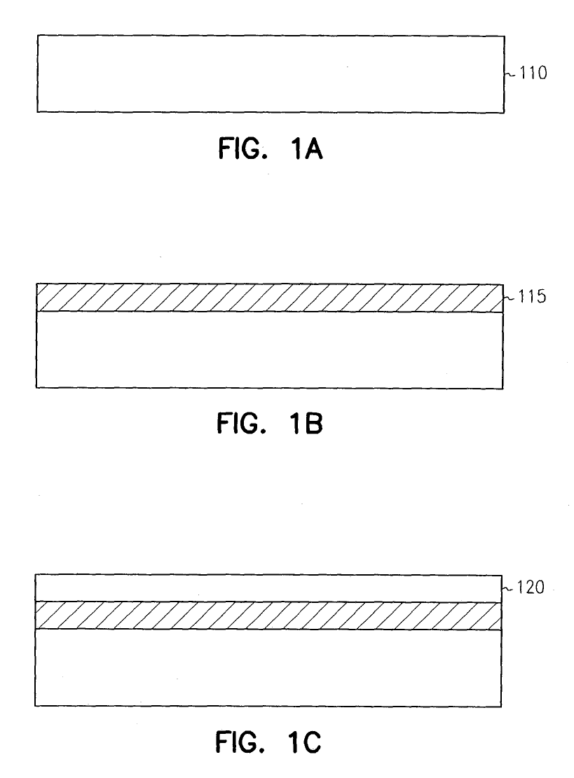

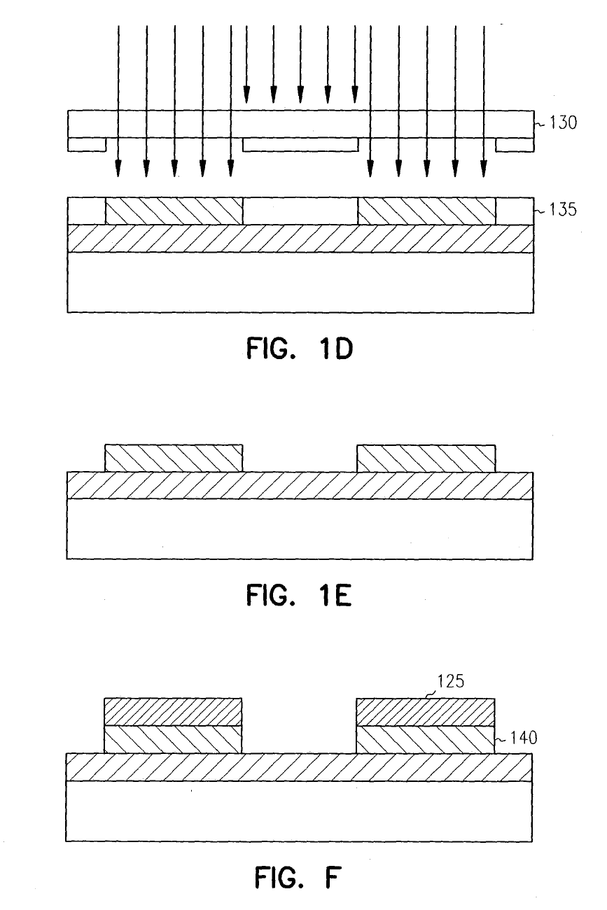

NeutrAvidin-biotin patterning is performed as illustrated in FIGS. 1A, 1B, 1C, 1D, 1E, 1F and 1G by applying a series of layering steps on top of a silicon substrate 110, including silane 115, photoactivatable biotin 120, NeutrAvidin 125, and biotinyated antibodies. This process uses a 3-aminopropylethoxysilane (3-APTS) that consequently forms a self-assembled monolayer (SAM). The SAM provides a uniform surface with exposed amine terminal groups to which the azide groups of NHS-ester conjugated biotin readily bind after UV irradiation. The NeutrAvidin-biotin bond is a very stable bond, Kₐ=1×10¹⁵ M⁻¹, that withstands a wide range of chemical and pH range variations. Avidin is a tetrameric molecule that has four binding sites for biotin. NeutrAvidin is a 60 kD molecule that is a refined form of avidin and that has less nonspecific binding to the substances than both avidin and streptavidin. Biotinylated molecules, such as antibodies are subsequently bound to the avidin through the biotin link.

An overview of the method of forming the patterning begins with a substrate 110 as illustrated in FIG. 1A. A layer of silane 115 is applied in FIG. 1B. A photobiotin coating 120 is spun on in FIG. 1C, and a chrome plated quartz mask 130 as shown in FIG. 1D is used with a positive tone exposure in FIG. 1E to form the pattern. Unexposed photosensitive material 135 is removed by deionized water as indicated in FIG. 1F. A blocker 140 is applied in FIG. 1G along with avidin 125. In further embodiments, the mask may be any type of device that creates spacial modulation of energy that can be used to pattern a layer.

In one example embodiment, the silane solution 3-aminopropyltriethoxysilane (3-APTS), NeutrAvidin, Superblock blocking solution and EZ-Link™ Photoactivatable Biotin was purchased from Pierce (Rockford, Ill.). The wash solution contained phosphate buffered saline with 0.05% Tween 20 (PBST). The NeutrAvidin conjugated with Alexa 488 fluorescent dye was purchased from Molecular Probes (Eugene, Oreg.). Tap water was filtered to a resistivity of 18.2 Mohm-cm using a Milli-Q Millipore filtration system. Tween 20 from Aldrich Chemical Company, Inc. (Milwaukee, Wis.) was used as a surfactant to decrease nonspecific binding. CD26 developing solution came from Shipley.

In one embodiment, a chrome plated quartz mask is processed in a GCA PG3600F Optical Pattern Generator using a pattern designed with L-Edit software. The mask is developed in a chrome etchant for 2 minutes, washed with deionized water, and developed in Shipley CD26 solution for 2 minutes.

The silane solution is prepared in a 50-mL amber bottle using 0.5 mL of 3-aminopropaltriethoxysilane and 24.0 mL of acetone in nitrogen atmosphere glovebox to create a 2% silane solution. The silation step begins by cutting 1 mm diameter, 10 cm long capillary tubes from Fischer Chemicals into 2 cm pieces. They are cleaned in a Harrick Plasma Cleaner/Sterilizer PDC 3-G for 10 minutes. The tubes are removed and placed in 100° C. Milli-Q filtered water for 30 minutes. The glass tubes are nitrogen dried and swiftly inserted into the bottle containing silane solution and incubated for 30 minutes. The tubes are removed from the bottle, sonicated in acetone for 10 minutes, nitrogen dried, and baked in an oven at 90° C. for 30 minutes.

EZ-Link™ Photoactivatable Biotin is mixed with 0.5 mL Millipore water to produce a 1 mg/mL solution. Manipulations with Photoactivatable Biotin are carried out under a photographic safe light, in the dark or in any other manner to prevent premature exposure to light. 20 μL of photobiotin is pipetted into the glass tube tubes and dried in an oven for 2 hours at 37° C.

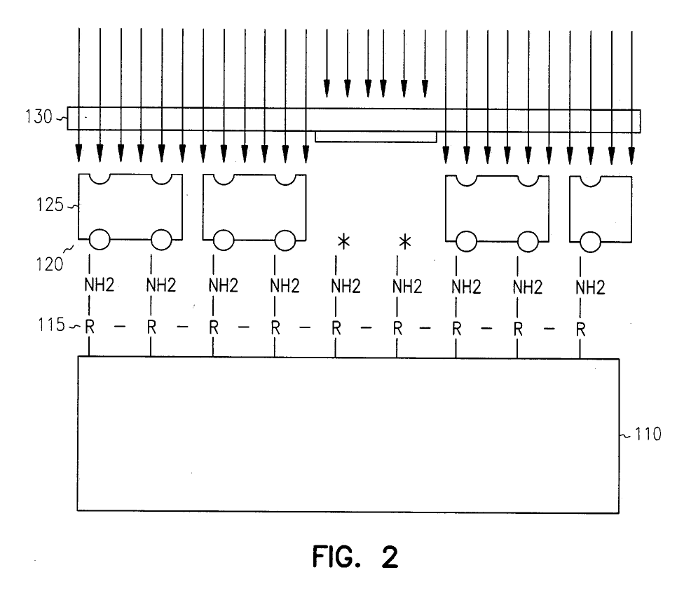

As seen in FIG. 2, the photobiotin-coated tubes are placed under a Hybrid Technology Group's (HTG) system 3HR contact/proximity mask aligner; the contact aligner is used in a flood exposure mode. The quartz mask is placed directly on the glass tubes and balanced evenly to ensure correct pattern transfer. The photobiotinylated tubes are exposed with UV light at 365 nm for 90 seconds, with an intensity of 15 mW/cm². The tubes are rinsed in PBST to remove unreacted photobiotin.

The tubes are incubated in PBST+2% BSA for 4 hours and washed 3× with PBST to block nonreactive sites. NeutrAvidin or NeutrAvidin conjugated with Alexa 488 dye (with 495 nm/519 nm excitation/emission) is prepared by reconstituting with Millipore filtered water (approximately 10 mg/mL in water) followed by dilution to 1 mg/mL into PBST. Each tube is incubated with 35 μL of NeutrAvidin to form layer 125 for 20 minutes. They are rinsed with PBST and blocked in PBST+2% BSA for 1 hour. The tubes are finally washed and stored in PBST bath until the beginning of the next step. When using the NeutrAvidin conjugated to Alexa 488 fluorescent dye, the tubes may be analyzed using a Zeiss microscope with a Omega Optical filter (450-490 nm/520 nm excitation/emission).

Areas that are not exposed have very low nonspecific binding of the Alexa-488 conjugated NeutrAvidin. The ease with which unexposed photoactivatable biotin is washed off contributes to the high patterning resolution possible with the photobiotin. The blocking agents in the Superblock solution bound to the newly exposed primary amine groups on the silane molecules. Blocking these amines minimized the nonspecific NeutrAvidin binding to these areas.

Different exposure durations may be used to determine the ideal amount of time required for activating the photobiotin using the HTG. Some durations are from approximately 30 seconds to 15 minutes. Ninety seconds was used in one embodiment. The intensity of the Alexa-488 fluorescence was diminished for shorter periods and the same of longer periods.

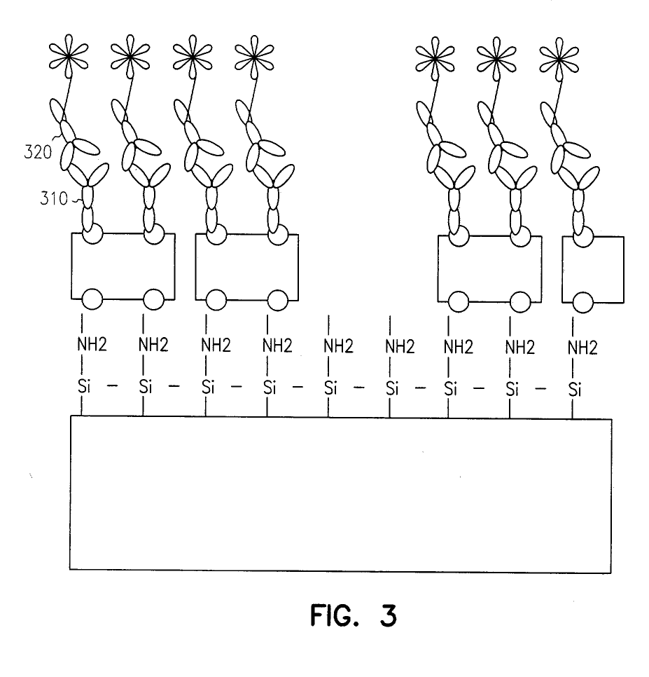

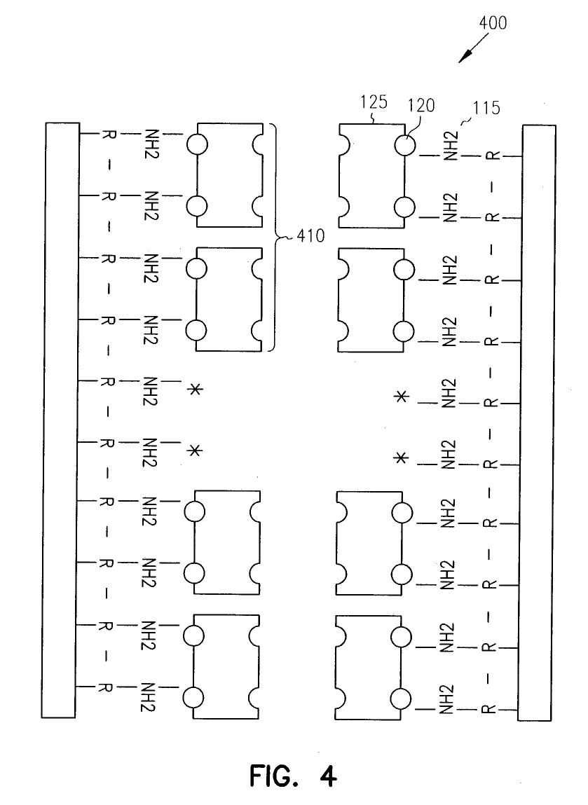

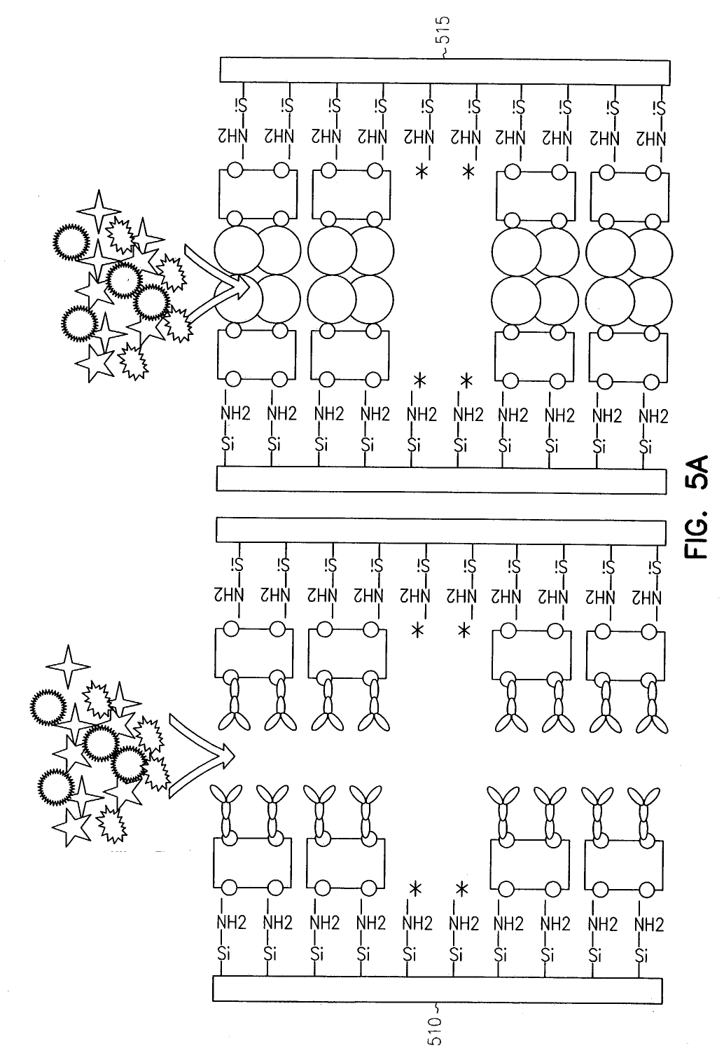

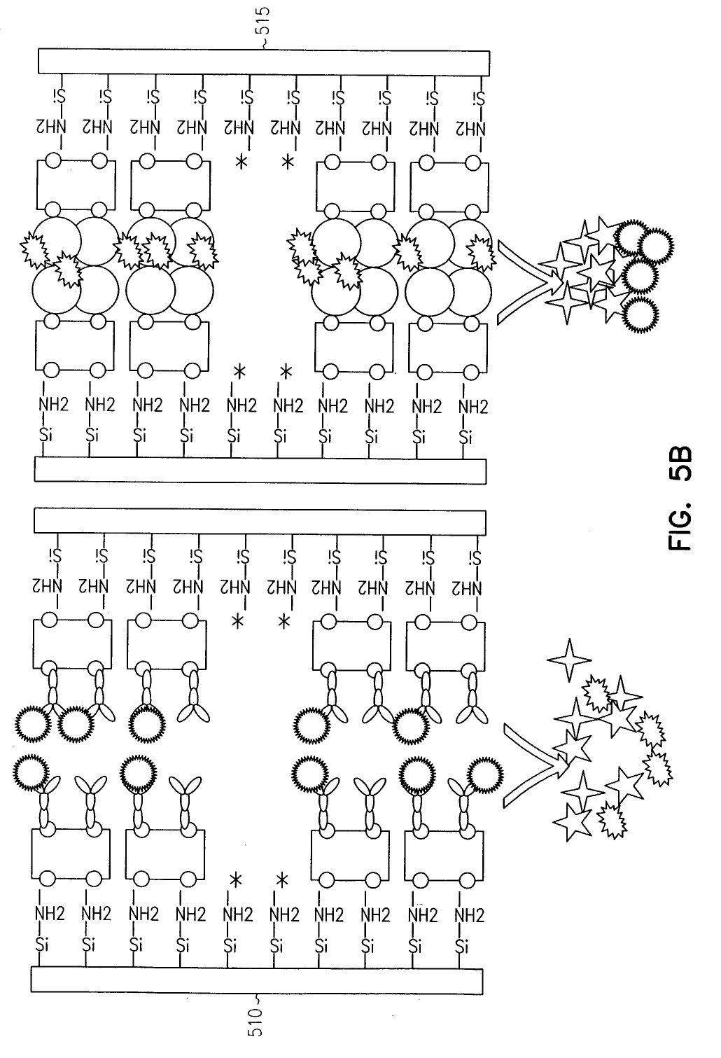

Once a molecule is biotinylated, it is able to be attached, as indicated in layer 310 in FIG. 3, to the inside of the capillary tube as long as steric hindrance or surface geometry does not prevent binding. In one embodiment, fluorescently labeled primary antibodies and protein-A coated spheres and E. coli cells server as model target antigens 320 coupled to the Aviden 310. FIG. 4 illustrates one tube 400 so patterned with silane 115, photo-biotin 120 and NeutrAvidin 125. In one embodiment, the tube 400 is a glass capillary tube, and bands 410 of NeutrAvidin are approximately 50 um, and are spaced approximately 25 um apart. formed on the tube

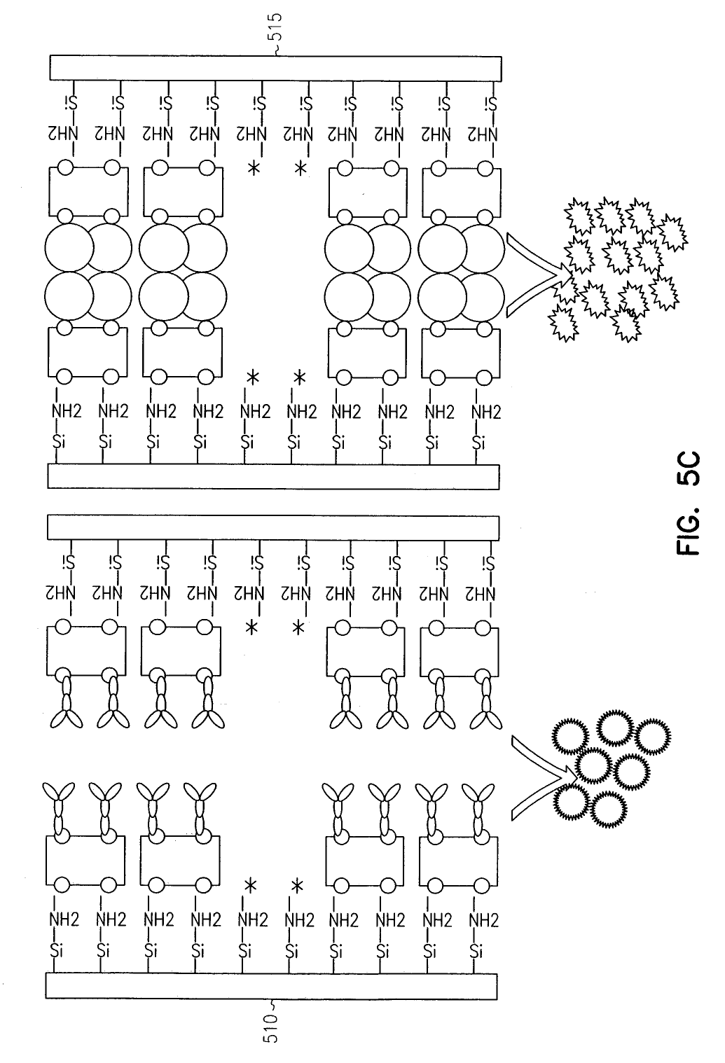

FIGS. 5A, 5B, and 5C illustrate nanofluidic affinity chromatography that is possible by incorporating the protein patterning technique to existing nanofluidic systems. The left side of each figure illustrates an antibody-based affinity column 510 while the right side illustrates a porous bead-based affinity column 515. The highly specific antibody-based column will bind to the antigen's surface epitopes 520 as indicated in FIG. 5B. The porous bead-based column will bind antigen by size of the antigen. The antibody of the target antigen can be adhered to the fluidic channel wall as seen in FIG. 5A. When a mixed solution, such as whole blood, serum, or contaminated solution, is added to the column, the antibodies or bead will bind to the target antigen or particles FIG. 5B. The adhered particulate will elute when rinsed with the proper pH buffer wash solution is added in FIG. 5C. A salty, or changed pH solution provides a less optimal condition for bonding, causing the adhered particulate to elute. The supernatant may be tested with standard ELISA protocols to calibrate the affinity chromatography system.

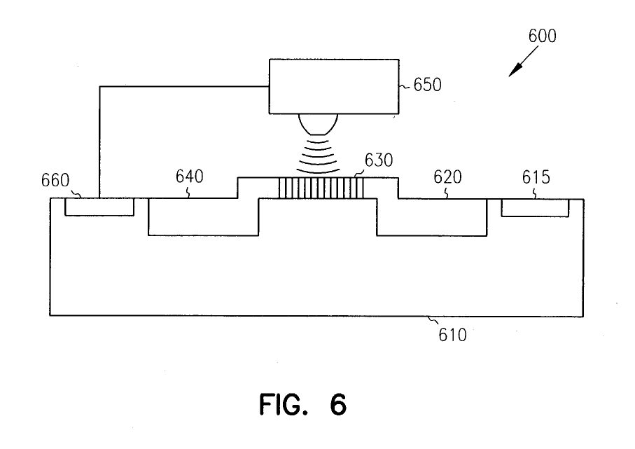

A micro or nano-fluidic system is shown at 600 in FIG. 6. A substrate, such as a silicon substrate 610 supports fluidics 615, which may comprise one or more series of sensors, pumps, passages and other small devices which may formed in or supported by the substrate 610. In one embodiment, the fluidics 615 are coupled to an input reservoir 620 for holding a biological sample. The biological sample is provided to a patterned tube 630 formed in accordance with the present invention. An output reservoir is coupled to the other end of tube 630 to collect samples and other solutions flowing through the tube.

In one embodiment, the tube 630 is supported on top of the substrate 610. In further embodiments, tube 630 is supported above the substrate, and may be bent to couple to reservoirs 620 and 640. A sensor 650, such as a biosensor or chromatography system is provided proximate the tube 630 to measure samples captured in the bands of the tube 630. The sensor 650 may be coupled directly to circuitry formed in or supported by the substrate 610 as indicated at 660, or may be coupled to further separate electronics for capturing data related to such measurements. In yet further embodiments, the sensor 650 is directly formed in or supported by the substrate.

In a further embodiment, different parameters were utilized for patterning a silicon surface. Reagents. Silane solution 3-aminopropyltriethoxysilane (3-APTS), avidin, 0.5 mg EZ-Link™ Photoactivatable Biotin, sodium meta-periodate, 5 mL dextrose desalting columns, sodium acetate, and biocytin hydrazide were purchased from Pierce (Rockford, Ill). Polyclonal, goat anti-mouse IgG antibodies and biotinylated goat anti-E.coli O157:H7 antibodies were purchased from Kirkegaard & Perry Laboratories (KPL, Gaithersburg, Md.). The biotinylated, polyclonal goat anti-rabbit antibodies, avidin conjugated with Alexa-488 fluorescent dye, and the protein A, FITC-labeled 40 nm FluoSpheres® were purchased from Molecular Probes (Eugene, Oreg.). The antibodies were diluted in phospate buffered saline with 0.1% Tween 20 (PBST). Tween 20 from Aldrich Chemical Company, Inc. (Milwaukee, Wis.) was used as a surfactant to decrease nonspecific binding. Tap water was filtered to a resistivity of 18.2 MΩ-cm using a Milli-Q Millipore filtration system. E.coli were cultured essentially as described by St. John. The wash solution contained PBST to provide a buffered solution that kept the E.coli cells intact and prevented protein degredation. CD26 developing solution and S1813 photoresist was obtained from Shipley, Inc.

Development of Microfabricated Pattern. A photoresist coated 4″ chrome plated quartz mask was processed in a GCA PG3600F Optical Pattern Generator to expose a pattern designed with L-Edit software. The mask was developed using standard photolithograhic methods.

Silanization of Silicon Wafer Surface. A 258 nm +/−5 nm oxide layer was grown on the surface of 3″ n-type (100) silicon wafers from Silicon Quest International (San Jose, Calif.) by treating with pyrogenic steam +4% Trans-PC (Dichloroethane) in a Thermco tube furnace for 45 minutes at 900° C.

The silane solution was prepared in a 50-mL amber bottle using 0.5 mL of 3-aminopropaltriethoxysilane and 24.0 mL of acetone in a nitrogen purged glovebox to create a 2% silane solution. The silanization step began by cleaning 2 cm² silicon chips in a Harrick Plasma Cleaner/Sterilizer PDC 3-G for 10 minutes. The chips were removed and placed in 100° C. Milli-Q filtered water for 30 minutes. The silicon chips were nitrogen dried then quickly inserted into the bottled silane solution and incubated in a closed container for 30 minutes. The chips were removed, sonicated in acetone for 10 minutes, nitrogen dried, and baked on a hotplate at 120° C. for 5 minutes.

Patterning of Silicon Wafer Surface. EZ-Link™ Photoactivatable biotin (Pierce, 0.5 mg) was mixed with 0.5 mL Millipore water to produce a 1 mg/mL solution. All manipulations with Photoactivatable Biotin were carried out under dark room conditions. 20 μL of photobiotin were pipetted onto the silicon chips, covered with glass cover slips from Fisher Scientific (Pittsburgh, Pa.) and dried in an oven for 2 hours at 37° C.

Pattern Transfer. The photobiotin-coated chips were placed under the Hybrid Technology Group's (HTG) system 3HR contact/proximity mask aligner; the contact aligner was used in the flood exposure mode. The quartz mask was placed directly on the silicon chips and balanced evenly to ensure correct pattern transfer. The photobiotinylated chips were exposed with UV light at 365 nm for 4 minutes, at intensity of 15 mW/cm². The chips were rinsed in PBST for 30 seconds to remove any unreacted photobiotin.

Blocking of Nonreactive Sites. The chips were incubated in Pierce's Superblock blocking solution for 1 hour and washed 3× in PBST.

Avidin Application. Solutions of avidin conjugated with Alexa-488 dye (with 495 nm/519 nm excitation/emission) was prepared by reconstituting ˜10 mg/mL with Millipore filtered water followed by dilution to 1 mg/mL in PBST. The reconstituted product was stored at 4° C. Each chip was incubated with 35 μL of avidin for 20 minutes. They were rinsed with PBST and dipped into Superblock solution. These blocking steps were repeated two times. The chips were finally washed and stored in a PBST bath until the next step. Samples treated with Alexa-488 conjugated avidin were analyzed using a Zeiss microscope with an Omega Optical filter (450-490 nm/520 nm excitation/emission).

Labeling and Biotinylating Anti-E.coli Antibodies. Goat anti-E.coli O157:H7 antibodies (Pierce) were labeled using the Alexa-594 protein labeling kit from Molecular Probes (590 nm/619 nm excitation/emission). The labeled antibodies were biotinylated with biocytin hydrazide. 300 μL of 3 mM sodium meta-periodate solution (Pierce) were added to 600 μL of the antibody solution. The solution was incubated in the dark for 30 minutes at room temperature to produce aldehyde groups from the carbohydrates. Excess sodium periodate was removed with a 5 mL desalting column (Pierce) that had been pre-equilibrated with 100 mM sodium acetate, pH 5.5. The fractions were collected and the absorbance of the fractions was measured in a spectrophotometer. The fractions containing high protein concentrations were pooled. 300 μl of 5 mM biocytin hydrazide solution was added to the pooled fractions and incubated for 1 hour at room temperature. The reaction was terminated by adding 200 μL of 0.1 M Tris stop solution. Unreacted biocytin hydrazide was removed by further desalting and the sample was brought to its original volume in stop solution.

Secondary Antibody Analysis of Anti-E.coli Antibodies. Avidin coated silicon chips were flooded with biotinylated, polyclonal goat anti-E.coli O157:H7 antibody, incubated for 20 min, and then washed repeatedly with PBST. Secondary rabbit anti-goat antibody conjugated to Texas Red (50 μg/mL working dilution; Pierce) was then added and the chips were incubated an additional 20 min prior to washing. Antibody binding was analyzed using a Zeiss microscope equipped with fluorescence optics (590-640 nm/620 nm excitation/emission).

Fluorescent Sphere Application. Biotinylated rabbit anti-goat antibodies were purchased in solution and were later diluted to 50 μg/mL. 35 μL of the biotinylated antibody solution was pipetted onto the avidin coated silicon chips. The chips were incubated for 20 minutes at room temperature. The chips were rinsed with PBST to remove any unreacted biotin and left in PBST solution until the next step. The 0.4 mL stock solution of protein A-labeled nanospheres (40 nm; yellow-green fluorescent; 505 nm/515 nm excitation/emission) was diluted to produce a working concentration of spheres ranging from 1×10⁷ to 1×10⁴ spheres/mL. 100 μL of sphere solution was pipetted onto each silicon chip, incubated for 20 minutes at room temperature, washed with PBST and dried with a low velocity nitrogen airstream. The chips were viewed in bright-field mode in a Zeiss microscope using a fluorescence filter Omega Optical filter (450-490 nm/520 nm excitation/emission).

Fluorescence Intensity Measurement. A Hamamatsu photomultiplier tube (PMT) detection assembly, HC 124-02, was used to detect the light intensity of the fluorescence coming from the patterned substrates. An Olympus IX70 inverted microscope with 20× and 40× objectives was used to visualize the samples. Imaging software was used to interpret the data collected from the PMT detection assembly.

### Conclusion

The use of a light activatable molecule, such as photoactivatable avidin-biotin is a simple and economical way to transfer micrometer scale patterns to the inner surface of a tube. Using ultraviolet light in conjunction with photolithographically patterned masks offers a method to derivitize biological molecules to the inside of glass tubes. Once the inner surface is patterned with avidin, biotinylated molecules or other biological molecules and cells can also be attached to the inner surface of the tube. Affinity chromatography can be realized at the nanofluidic level with this technique. Photoactivatable biotin has a 533.36 MW and is 3 nm in length. Therefore, a patterning resolution below 10 nm may be realized. Further forms of photobiotin, such as photoactivatable biotin (a nitro(aryl)azide derivative of biotin, MW 533.65, 3 nm long), photocleavable biotin, (NHS-Iminobiotin, MW 421.32, 1.35 nm long), and caged biotin (N-(4-azido-2-nitrophenyl)-N-(3-biotinylaminopropyl)-N-methyl-1-3-propanediamine), and others which can be used to label proteins and nucleic acids. The patterning of biotin, Neutravidin, and biotinylated antibodies may also be done on a planar substrate as well as the binding of protein A-coated microspheres to biotinylated antibodies.

Other materials that covalently bind to an organic surface when exposed to UV light may also be utilized. The patterning methods may also be compatible with other surfaces including nanofluidic tubes in glass, silicon, and plastic.

In further embodiments, selective, spatial pattering of materials inside enclosed micro- or nanochannels utilizes light, X-ray radiation, UV radiation, electron beam and other directed energy, and magnetic energy that photoactivates, uncages, photolyses polymerizes, crosslinks, degrades, creates free radicals, dextrorotation, and levorotation different activatable materials. Such photoactivatable reagents comprise photoactivatable biotin, neurotransmitters, nucleotides, phosphates, GFP, ABH, (p-Azidobenzoyl hydrazide, a carbohydrate-reactive photoactivatable cross-linker), and Sulfo-SANPAH (N-Sulfosuccinimidyl-6-[4′-azido-2′-nitrophenylamino] hexanoate).

The energies allow materials encountering the energies to be selectively and/or spatially patterned whereas materials not encountering these energies bind to a lesser degree or not at all to the inside of the channel. The use of magnetic energy may modify the materials in a way that allows materials to be temporarily suspended within the enclosed channel while a magnetic field is present. In the absence of said magnetic field the materials, if they have not be otherwise altered in a way to bind them to the surface, may be removed from the channel. Combinations of said energies may be used to offer a variety of methods for patterning, such as the use of suspending materials (material A) with a magnetic and biological (i.e. enzymatic) reagent in a region where materials (material B) modifiable by light or other energies may interact with the biological component. Consequently, the material B in the region of the spatially constrained material A may be selectively patterned.

Targets of patterned biotin are avidin, streptavidin, or Neutravidin which could subsequently capture biotinylated reagents. Avidin biotin patterning in micro- or nanochannels can be modeled with a capillary tube. The capillary tube is novel, stable, and economical patterning method for adhering proteins to the inner surface of micro- and nanofluidic systems. In one embodiment, biotinylated reagents comprise biotinylated proteins to include antibodies that can capture target antigens. Biotinylated reagents comprise biotinylated microspheres that may be porous to bind molecules by size or coated with a secondary molecule to capture a tertiary molecule by affinity binding.

These methods can be used for affinity chromatography within enclosed micro- or nanochannels and to separate molecules in heterogeneous or homogeneous solution mixtures comprising blood, environmental samples, biological warfare samples, and airborne samples. Separated molecules may be eluted from the micro- or nanochannel. Elution techniques comprise changes in salinity, pH, electrophoretic potential. Molecules may be bound through a silane layer or a crosslinker. The silane layer comprises 3-aminopropyltriethoxysilane in one embodiment. The elution target can be the captured secondary molecule or the primary molecule bound to the substrate (with or without the silane linker).

One of the potential benefits of various embodiments of the invention are a reduction in the required solution quantities from the microliter range to at least as small as nanoliter volume. Calibration may be performed using antibody and porous bead affinity chromatography systems with enzyme linked immunosorbant assay (ELISA) protocols. Furthermore, this technique will be applied to micro- and nanofluidic systems in silicon, glass, and plastic. The channels may be made of silicon containing substrate or polymer containing substrate in further embodiments.

1. A method of creating a desired biosample affinity in a glass tube, the method comprising:
applying a photo activatable biotin to the inside of the glass tube;
exposing the photo activatable biotin to light through a mask having a desired pattern; and
removing unreacted photo activatable biotin.

2. The method of claim 1 and further comprising coating the inside of the glass tube with silane prior to applying the photo activatable biotin.

3. The method of claim 1 and further comprising:
applying a blocker to the patterned photo activatable biotin; and
binding avidin to the patterned photo activatable biotin.

4. The method of claim 1 wherein the light has a wavelength in the UV range.

5. The method of claim 4 wherein the light has a wavelength of approximately 350 to 365 nm.

6. The method of claim 4 wherein the biotin is exposed to the light for approximately 90 seconds.

7. The method of claim 1 wherein the mask comprises a chrome plated photomask.

8. A method of creating affinity in capillary tubes, the method comprising:
coating the enclosed structure in a silane solution;
applying a photoactivatable Biotin material to the inside of a capillary tube;
exposing the enclosed structure to UV light through a mask having a desired pattern;
removing unreacted photoactivatable Biotin; and
incubating the tube with avidin.

9. The method of claim 8 and further comprising binding model target antigens to the avidin.

10. The method of claim 9 wherein the antigens comprise antibodies or protein coated spheres or E.coli cells.

11. The method of claim 9 wherein the antigens are biotinylated.

12. A method of creating biosample affinity in enclosed structures, the method comprising:
coating the enclosed structure;
applying a photo activatable material to the enclosed structure;
exposing the enclosed structure to UV light through a mask having a desired pattern; and
removing unreacted photo activatable material.

13. A method of creating a pattern having biosample affinity on a silicon substrate, the method comprising:
applying a photo activatable biomolecule supported by the substrate;
exposing the substrate to light through a mask having a desired pattern; and
removing unreacted photo activatable biomolecule.

14. A method of creating a pattern having biosample affinity on a surface, the method comprising:
applying a silane layer supported by the substrate;
applying a photo activatable biomolecule supported by the silane layer;
exposing the layers to light through a mask having a desired pattern; and
removing unreacted photo activatable biomolecule.

15. The method of claim 14 and further comprising binding model target antigens to the photo activatable biomolecule.

16. The method of claim 15 wherein the antigens comprise antibodies or protein coated spheres or E.coli cells.

17. The method of claim 15 wherein the antigens are biotinylated.

18. A container comprising:
an inner and outer surface;
a silane layer supported by the inner surface of the tube;
a patterned photoactivatable biotin layer supported by the silane layer; and
an avidin layer bound to the biotin layer.

19. The container of claim 20 wherein the silane layer comprises 3-aminopropyltriethoxysilane and the biotin layer comprises N-hydroxysuccinimide ester of photoactivatable biotin.

20. A small fluidic system comprising:
a substrate having structures for handling biosamples; and
a tube supported by the substrate and coupled to the structures, the tube having patterned immobilized functional proteins on an inner surface.

21. The fluidic system of claim 20 and further comprising an input reservoir coupled to an input of the tube, and an output reservoir coupled to an output of the tube.

22. The fluidic system of claim 20 wherein the tube comprises:
an inner and outer surface;
a silane layer supported by the inner surface of the tube;
a patterned photoactivatable biotin layer supported by the silane layer; and
an avidin layer bound to the biotin layer.

23. The fluidic system of claim 20 wherein the tubes comprise micro or nano-tubes formed in a silicon containing substrate or a polymer containing substrate.

24. A method of creating a desired biosample affinity in an enclosed channel, the method comprising:
applying an energy activatable reagent to the inside of the channel;
exposing the energy activatable reagent to energy through a mask having a desired pattern to modify binding properties of the energy activatable reagent; and
removing unexposed energy activatable reagent.

25. The method of claim 24 wherein the energy is selected from the group consisting of light, X-ray radiation, UV radiation, electron beam and other directed energy, and magnetic energy.

26. The method of claim 24 wherein the reagent is selected from the group consisting of photoactivatable biotin, neurotransmitters, nucleotides, phosphates, GFP, ABH, (p-Azidobenzoyl hydrazide, a carbohydrate-reactive photoactivatable cross-linker), and Sulfo-SANPAH (N-Sulfosuccinimidyl-6-[4′-azido-2′-nitrophenylamino] hexanoate).

27. The method of claim 24 wherein the binding properties are modified in a manner that photoactivates, uncages, photolyses polymerizes, crosslinks, degrades, creates free radicals, dextrorotation, or levorotation.

28. The method of claim 24 wherein the energy comprises magnetic energy that causes materials to be temporarily suspended in the channel.

29. The method of claim 24 wherein the reagent comprises photoactivatable reagents or light sensitive reagents.

30. The method of claim 24 wherein reagents with modified binding properties interact with a biological component.

31. The method of claim 24 wherein the reagent comprises photoactivatable biotin having a target comprising avidin, streptavidin or Neutravidin.

32. The method of claim 31 wherein the target captures biotinylated reagents.

33. The method of claim 32 wherein the biotinylated reagents comprise biotinylated proteins or antibodies.

34. The method of claim 32 wherein the biotinylated reagents comprise biotinylated microspheres.

35. The method of claim 34 wherein the biotinylated microspheres are porous to bind molecules by size or coated with a secondary molecule to capture a tertiary molecule by affinity binding.

36. The method of claim 35 wherein the various biotinylated microspheres are used in affinity chromatography to separate molecules.

37. The method of claim 36 and further comprising eluting the separated molecules from the channel.

38. The method of claim 37 wherein eluting the separated molecules from the channel comprises changing salinity, pH, or electrophoretic potential.
